(19) United States
(12) Patent Application Publication
(10) Pub. No.: US 2024/0412015 A1
(43) Pub. Date: Dec. 12, 2024

(54) IMAGE PROCESSING APPARATUS, IMAGE CAPTURE APPARATUS, SYSTEM, AND CONTROL METHOD

(71) Applicant: CANON KABUSHIKI KAISHA, Tokyo (JP)

(72) Inventor: Takuya KUBO, Kanagawa (JP)

(21) Appl. No.: 18/675,240

(22) Filed: May 28, 2024

(30) Foreign Application Priority Data
Jun. 6, 2023 (JP) 2023-093305

(51) Int. Cl. G06K 7/10; G06K 7/14; G06K 19/06; G06T 7/00; G16H 10/60; H04N 7/18

(52) U.S. Cl. CPC G06K 7/10722; G06K 7/1413; G06K 19/06028; G06T 7/0012; G16H 10/60; H04N 7/183; G06T 2200/24; G06T 2207/30036

(57) ABSTRACT

An image processing apparatus obtains, from a first external apparatus, a first image in which a predetermined part of a patient has been captured; and in a case where information of the patient cannot be obtained from the first image, transmits, to a second external apparatus, a second image for identifying a patient, the second image having been added to the first image, and obtains, from the second external apparatus, information of the patient corresponding to the second image and associate the information with the first image.

### BACKGROUND OF THE INVENTION

### Field of the Invention

The present invention relates to a system that manages an affected part image and patient information in association with each other.

### Description of the Related Art

Japanese Patent Laid-Open No. 2015-219699 describes a system that manages an affected part image and patient information in association with each other. Further, Japanese Patent Laid-Open No. 2015-219699 describes obtaining identification information (patient information) of a patient from a barcode, associating it with an affected part image in which an affected part is captured, and transmitting the patient information and the affected part image to an information processing apparatus via a network.

In a conventional system, when an image capture apparatus transmits patient information to an information processing apparatus by wireless communication, transmission can be performed properly in a good communication environment, but transmission may fail in a poor communication environment. When the patient information has not been successfully transmitted, an affected part image will be stored without being associated with the patient information.

For example, in a case where one day's worth of affected part images are collectively transmitted to the information processing apparatus, when affected part images that have been associated with patient information and affected part images that have not been associated with patient information are mixed, the affected part images that have not been associated need to be sorted and associated with patient information, which requires a great amount of time and effort.

### SUMMARY OF THE INVENTION

The present invention has been made in consideration of the aforementioned problems, and realizes a technique that makes it possible to, even in a case where images that have been associated with patient information and images that have not been associated with patient information are mixed, reduce the work of sorting the images that have not been associated and associating them with patient information.

In order to solve the aforementioned problems, the present invention provides an image processing apparatus comprising: an obtaining unit that obtains, from a first external apparatus, a first image in which a predetermined part of a patient has been captured; and a control unit that: in a case where information of the patient cannot be obtained from the first image, transmits, to a second external apparatus, a second image for identifying a patient, the second image having been added to the first image, and obtains, from the second external apparatus, information of the patient corresponding to the second image and associate the information with the first image.

In order to solve the aforementioned problems, the present invention provides an image capture apparatus comprising: an image capture unit that captures a first image indicating a predetermined part of a patient and a second image for identifying a patient; a communication unit that transmits the second image to an external apparatus; and a control unit that: in a case where information of the patient corresponding to the second image has been obtained from the external apparatus, associates the information of the patient with the first image, and in a case where the information of the patient corresponding to the second image cannot be obtained from the external apparatus, adds the second image to the first image.

In order to solve the aforementioned problems, the present invention provides a system including: an image capture apparatus that captures a first image indicating a predetermined part of a patient and a second image for identifying a patient; an image processing apparatus that obtains the first image to which the second image has been added from the image capture apparatus; and an information management apparatus that reads identification information of the patient from the second image received from the image capture apparatus or the image processing apparatus and transmits information of the patient corresponding to the identification information to the image capture apparatus or the image processing apparatus.

In order to solve the aforementioned problems, the present invention provides a method of controlling an image processing apparatus, the method comprising: obtaining, from a first external apparatus, a first image in which a predetermined part of a patient has been captured; and in a case where information of the patient cannot be obtained from the first image, transmitting, to a second external apparatus, a second image for identifying a patient, the second image having been added to the first image, and obtaining, from the second external apparatus, information of the patient corresponding to the second image and associating the information with the first image.

In order to solve the aforementioned problems, the present invention provides a method of controlling an image capture apparatus, the method comprising: capturing a second image for identifying a patient; transmitting the second image to an external apparatus; and in a case where information of the patient corresponding to the second image has been obtained from the external apparatus, associating the information of the patient with a first image in which the patient has been captured, and in a case where the information of the patient corresponding to the second image cannot be obtained from the external apparatus, adding the second image to the first image.

According to the present invention, it is possible to, even in a case where images that have been associated with patient information and images that have not been associated with patient information are mixed, reduce the work of sorting the images that have not been associated and associating them with patient information.

Further features of the present invention will become apparent from the following description of exemplary embodiments (with reference to the attached drawings).

### BRIEF DESCRIPTION OF THE DRAWINGS

FIG. 1 is a diagram of a configuration of a system according to the present embodiment.

FIGS. 2A and 2B are diagrams exemplifying a barcode and patient information according to the present embodiment.

FIG. 3 is a diagram exemplifying transition of screens of an image capture apparatus according to a first embodiment.

FIGS. 4A and 4B are flowcharts illustrating processing of the image capture apparatus and an information management apparatus according to the first embodiment.

FIG. 5 is a flowchart illustrating processing of the image capture apparatus according to the first embodiment.

FIGS. 6A and 6B are diagrams exemplifying a data configuration of an affected part image according to the first embodiment.

FIG. 7 is a flowchart illustrating processing of the image processing apparatus according to the first embodiment.

FIGS. 8A and 8B are diagrams exemplifying affected part images stored in the image capture apparatus according to the first embodiment.

FIGS. 9A to 9D are diagrams exemplifying transition of screens of the image processing apparatus according to the first embodiment.

FIGS. 10A and 10B are flowcharts illustrating processing of the image processing apparatus and the information management apparatus according to the first embodiment.

FIG. 11 is a diagram exemplifying affected part images managed by the information management apparatus according to the first embodiment.

FIGS. 12A and 12B are flowcharts illustrating processing of the image processing apparatus and the information management apparatus according to the first embodiment.

FIG. 13 is a flowchart illustrating processing of the image processing apparatus according to the first embodiment.

FIG. 14 is a flowchart illustrating processing of the image capture apparatus according to the first embodiment.

FIG. 15 is a diagram exemplifying affected part images stored in the image capture apparatus according to a second embodiment.

FIG. 16 is a diagram exemplifying transition of a screen of the image processing apparatus according to the second embodiment.

FIG. 17 is a flowchart illustrating processing of the image processing apparatus according to the second embodiment.

FIG. 18 is a flowchart illustrating processing of the image capture apparatus according to a third embodiment.

FIG. 19 is a diagram exemplifying affected part images stored in the image capture apparatus according to the third embodiment.

### DESCRIPTION OF THE EMBODIMENTS

Hereinafter, embodiments will be described in detail with reference to the attached drawings. Note, the following embodiments are not intended to limit the scope of the claimed invention. Multiple features are described in the embodiments, but limitation is not made to an invention that requires all such features, and multiple such features may be combined as appropriate. Furthermore, in the attached drawings, the same reference numerals are given to the same or similar configurations, and redundant description thereof is omitted.

### System Configuration

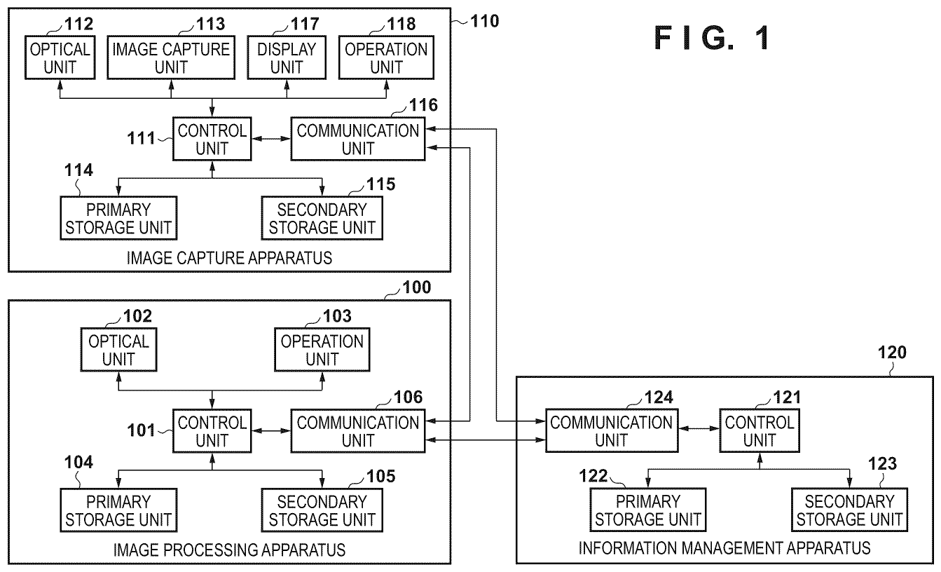

A configuration of a system according to the present embodiment will be described with reference to FIG. 1.

The system according to the present embodiment includes an image processing apparatus 100, an image capture apparatus 110, and an information management apparatus 120. The image processing apparatus 100, the image capture apparatus 110, and the information management apparatus 120 are connected so as to be capable of wired communication or wireless communication.

The image capture apparatus 110 is a digital camera or the like for a doctor or medical staff on a medical site to capture and record an image of a patient and to capture and record an image of a barcode assigned to a respective patient to identify the patient. The image capture apparatus 110 associates an affected part image with patient information and records the image, adds a barcode image to an affected part image and records the image, and uploads the affected part image associated with the patient information or the affected part image to which the barcode image has been added to the information management apparatus 120.

The image processing apparatus 100 is a portable terminal, such as a smartphone or a tablet computer, that reads patient information from a barcode image received from the image capture apparatus and transmits the read patient information to the image capture apparatus. The image processing apparatus 100 associates an affected part image with patient information and records the image, adds a barcode image to an affected part image and records the image, and uploads the affected part image associated with the patient information or the affected part image to which the barcode image has been added to the information management apparatus 120.

The information management apparatus 120 is a server computer that performs management, such as storage, update, and deletion, of an affected part image associated with patient information or an affected part image to which a barcode image has been added received from the image capture apparatus 110.

In the present embodiment, a configuration in which the image processing apparatus 100 is configured to be separate from the image capture apparatus 110 and is connected to the image capture apparatus 110 so as to be capable of communication will be described, but a configuration may be taken such that the image processing apparatus 100 and the image capture apparatus 110 are integrated.

Further, in the present embodiment, a case where the image capture apparatus 110 is a digital camera will be described, but it is not limited to a digital camera and may be a portable communication terminal, such as a smartphone or a tablet computer, or an information processing terminal.

### First Embodiment

First, a first embodiment will be described. In the first embodiment, an example in which the image capture apparatus 110 captures a barcode image for identifying a patient and an affected part image of the patient and associates patient information received from the information management apparatus 120 with the affected part image will be described. In the first embodiment, the affected part image associated with the patient information is transmitted to the image processing apparatus 100 and uploaded from the image processing apparatus 100 to the information management apparatus 120.

### Apparatus Configuration

Next, a configuration and functions of the image capture apparatus 110 will be described with reference to FIG. 1.

The image capture apparatus 110 captures a barcode image and an affected part image.

A control unit 111 includes a processor, such as a CPU, that performs computational processing and control processing related to the image capture apparatus 110. The control unit 111 realizes respective processes of flowcharts to be described later by executing programs stored in a secondary storage unit 115.

An optical unit 112 includes a group of lenses including a zoom lens and a focus lens, and a shutter that includes an aperture function.

An image capture unit 113 includes an image sensor constituted by a CCD, a CMOS, or the like, which converts a subject image into an electric signal, and an A/D converter that converts an analog image signal outputted from the image sensor into a digital signal. The image capture unit 113 converts light of the subject image, which has been formed by the lenses included in the image capture unit 113 under the control of the control unit 111, into an electric signal using the image sensor, performs noise reduction processing and the like, and outputs image data constituted by a digital signal.

A primary storage unit 114 is a volatile memory, such as a RAM, and constants and variables for operation of the control unit 111, programs read out from the secondary storage unit 115, and the like are loaded.

The secondary storage unit 115 is a non-volatile memory, such as a ROM, a flash memory, or a hard disk, and stores constants for operation of the control unit 111, programs (firmware), and the like. The programs according to the present embodiment include programs for executing flowcharts to be described later.

A communication unit 116 is connected to an external apparatus, such as the image processing apparatus 100 or the information management apparatus 120, so as to be capable of wired communication or wireless communication and transmits and receives data, such as an affected part image, a barcode image, and patient information. The communication unit 116 can be connected to a wireless local area network (LAN) and the Internet. The communication unit 116 is not limited to a wireless LAN, and a wireless communication interface (e.g., infrared communication, Bluetooth®, Bluetooth® Low Energy, and wireless USB) or a wired connection interface (e.g., USB cable, HDMI®, and IEEE 1394) may be used. In the present embodiment, assume that the image capture apparatus 110 and the image processing apparatus 100 are connected so as to be capable of wired communication, and the image capture apparatus 110 and the information management apparatus 120 are connected so as to be capable of wireless communication.

A display unit 117 performs display of images, display of characters for interactive operation, and the like. The display unit 117 is a display device, such as a liquid crystal display or an organic EL display.

An operation unit 118 includes operation members, such as various switches, buttons, and a touch panel, that receive various operations from a user. The touch panel is formed to be integrated with the display unit 117.

Next, a configuration and functions of the image processing apparatus 100 will be described with reference to FIG. 1.

The image processing apparatus 100 is a client terminal that transmits an affected part image received from the image capture apparatus 110 to the information management apparatus 120.

The image processing apparatus 100 includes a control unit 101, a display unit 102, an operation unit 103, a primary storage unit 104, a secondary storage unit 105, and a communication unit 106. The basic functions of these elements are similar to those of the image capture apparatus 110, and so, a detailed description thereof will be omitted.

The secondary storage unit 105 stores an operating system (OS), which is basic software to be executed by the control unit 101, and an application that realizes an applied function in cooperation with the OS. Further, in the present embodiment, the secondary storage unit 105 stores an image management application for communicating with the image capture apparatus 110 and the information management apparatus 120.

The processing of the image processing apparatus 100 according to the present embodiment is realized by reading software provided by an application. Assume that the application includes software for using the basic functions of the OS installed on the image processing apparatus 100.

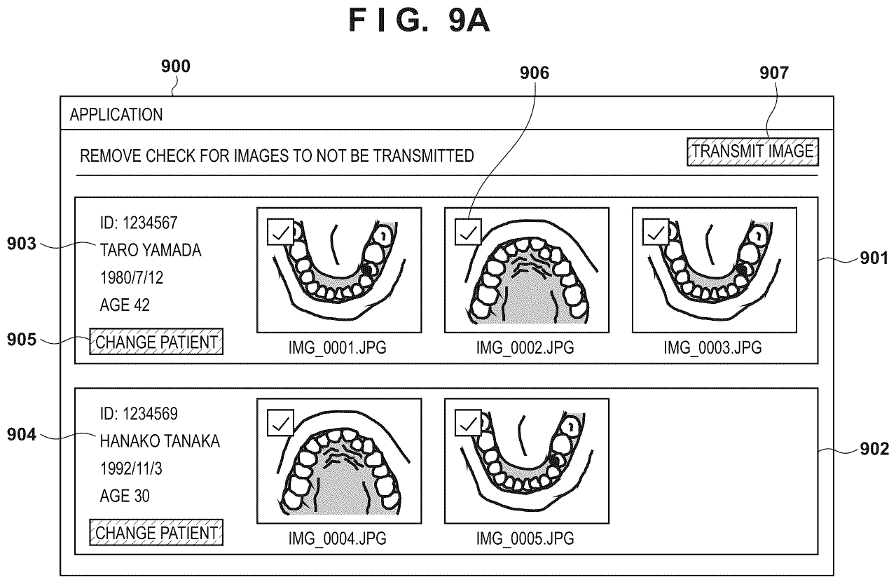

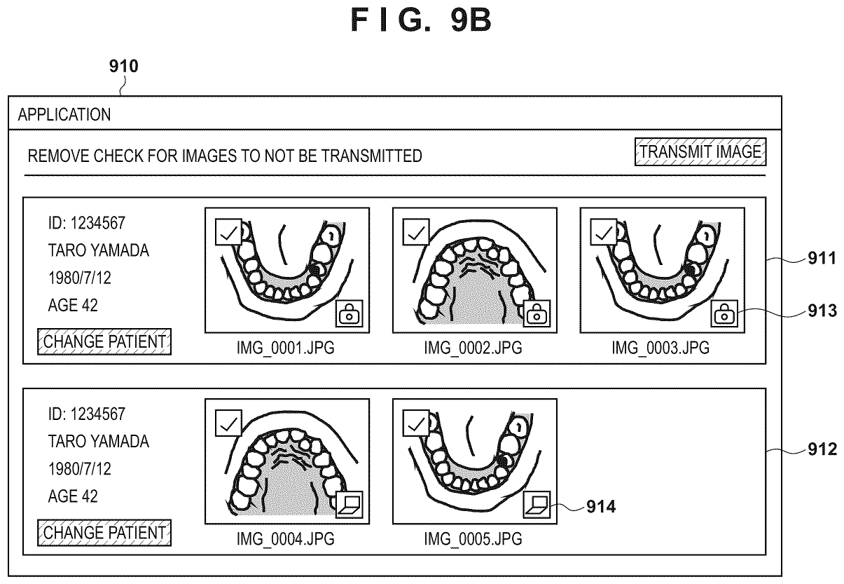

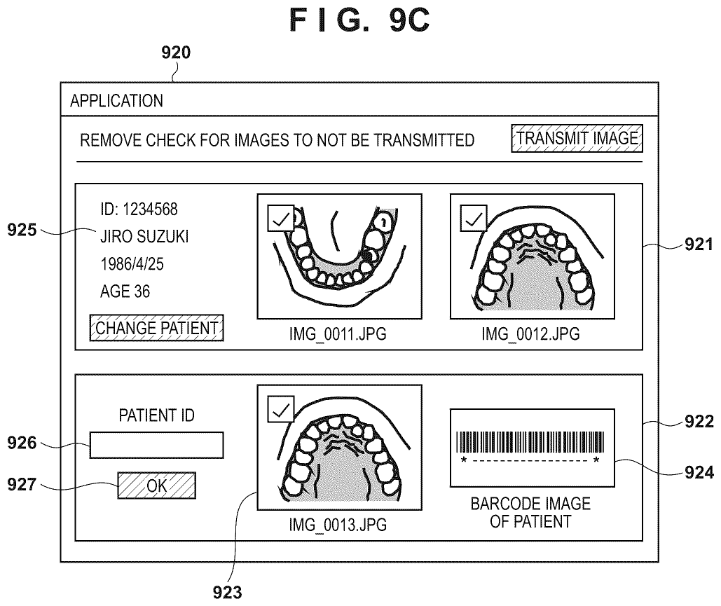

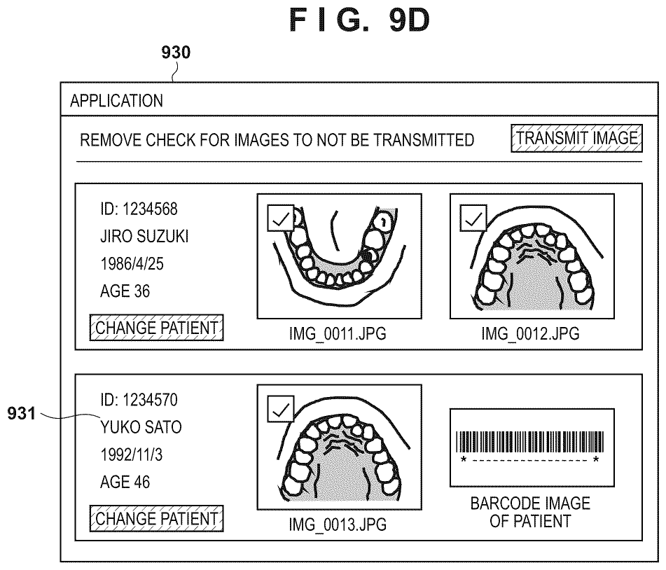

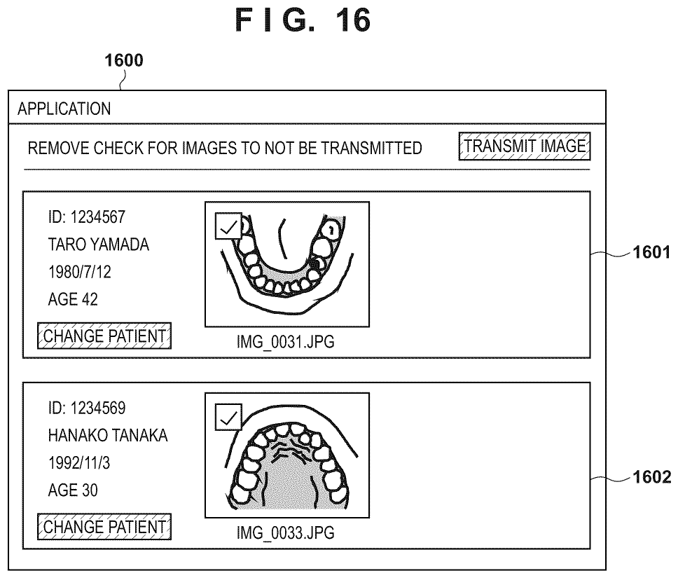

As will be described later in FIGS. 9 and 16, the image management application includes a function of searching for patient information from a barcode image received from the image capture apparatus 110, a function of associating an affected part image with patient information and recording the image, a function of adding a barcode image to an affected part image, and a function of uploading an affected part image associated with patient information or an affected part image to which a barcode image has been added to the information management apparatus 120.

Next, a configuration and functions of the information management apparatus 120 will be described with reference to FIG. 1.

The information management apparatus 120 includes a control unit 121, a primary storage unit 122, a secondary storage unit 123, and a communication unit 124. The basic functions of these elements are similar to those of the image capture apparatus 110 and the image processing apparatus 100, and so, a detailed description thereof will be omitted.

The information management apparatus 120 includes a function of receiving from the image processing apparatus 100 and managing an affected part image associated with patient information or an affected part image to which a barcode image has been added. Further, the information management apparatus 120 stores patient information corresponding to a barcode image, searches for patient information corresponding to a barcode image received from the image capture apparatus 110 or the image processing apparatus 100, and transmits the patient information to the image capture apparatus 110 or the image processing apparatus 100.

### Processing of Associating Affected Part Image With Patient Information

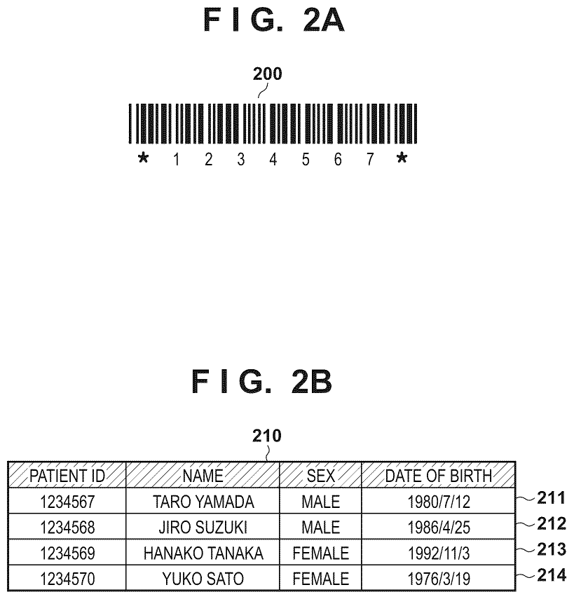

Next, processing for associating an affected part image with patient information according to the first embodiment will be described with reference to FIGS. 2A to 14 in addition to FIG. 1.

FIG. 2A illustrates a barcode to which patient information has been added.

On a medical site, a barcode to which unique identification information (patient ID) for a respective patient has been added may be used to identify the patient. For example, an inpatient may be wearing a wristband on which a barcode is printed, or a barcode may be printed on an outpatient reception form.

In the present embodiment, a barcode image for which a barcode 200 to which a patient ID has been added has been captured by the image capture apparatus 110 is transmitted to the information management apparatus 120, and patient information corresponding to the barcode image is received from the information management apparatus 120. The present invention is not limited to only a barcode (one-dimensional code), and for example, a two-dimensional code (e.g., QR code®) or biometric information (e.g., fingerprint or face recognition) may be used so long as it is information with which a patient can be identified.

FIG. 2B illustrates patient information managed by the information management apparatus 120. In the example of FIG. 2B, four people's patient information 211 to 214 are registered in patient information 210. The patient information 211 to 214 are uniquely identified by patient IDs and include patient attribute information, such as name, sex, and date of birth.

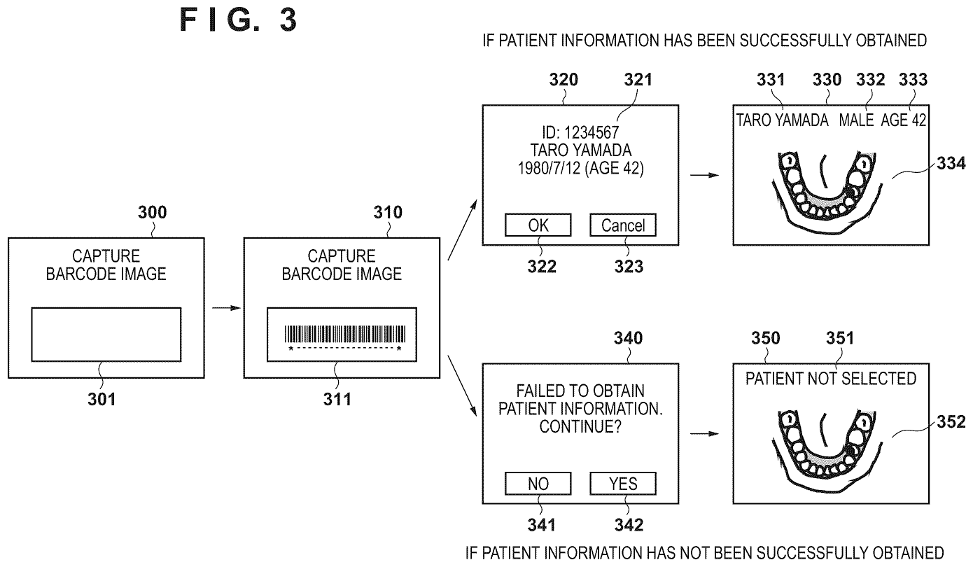

Next, screens to be displayed on the display unit 117 of the image capture apparatus 110 when the image capture apparatus 110 captures a barcode image and obtains patient information from the information management apparatus 120 will be described with reference to FIG. 3.

A screen 300 illustrates an initial screen for capturing a barcode image. The screen 300 is a live view screen, and a user can capture an image by aligning a barcode with a frame 301 of the screen 300. A screen 310 illustrates a screen while capturing an image of a barcode. When the user gives an image capturing instruction while a barcode is displayed in a frame 311 of the screen 310, the image capture apparatus 110 captures a barcode image and transmits it to the information management apparatus 120. When the information management apparatus 120 successfully obtains patient information based on the barcode image obtained from the image capture apparatus 110, the image capture apparatus 110 will receive the patient information from the information management apparatus 120 and transition to a screen 320, which displays patient information 321. The user can cause transition to a screen 330 by operating an “OK” button 322 when they determine that the patient information 321 displayed on the screen 320 is correct and capture an affected part image. The user can cause transition to the screen 300 by operating a “Cancel” button 323 when they determine that the patient information 321 displayed on the screen 320 is incorrect and re-capture a barcode image. The screen 330 is a live view screen, and patient information 331 to 333 obtained from the information management apparatus 120 and a live view 334 in which an affected part of a patient corresponding to that patient information is captured are displayed.

When the information management apparatus 120 fails to obtain patient information, it is conceivable, for example, that a barcode image cannot be transmitted from the image capture apparatus 110 to the information management apparatus 120 due to a wireless communication radio signal not being strong enough or the radio signal not reaching or that the information management apparatus 120 has failed to analyze the barcode image. When the information management apparatus 120 has failed to obtain patient information, the image capture apparatus 110 does not receive patient information from the information management apparatus 120, and thus, the screen 310 transitions to a screen 340. On the screen 340, a message indicating that patient information has not been successfully obtained from the information management apparatus 120 and prompting the user to confirm whether to continue capturing of an affected part image are displayed. When patient information cannot be obtained from the information management apparatus 120, the user can cause transition to a screen 350 by operating a “Yes” button 342 and capture an affected part image or can cause transition to the screen 300 by operating a “No” button 341 and re-capture a barcode image. On the screen 350, a state 351 that patient information has not been obtained and a live view 352 of an affected part image are displayed.

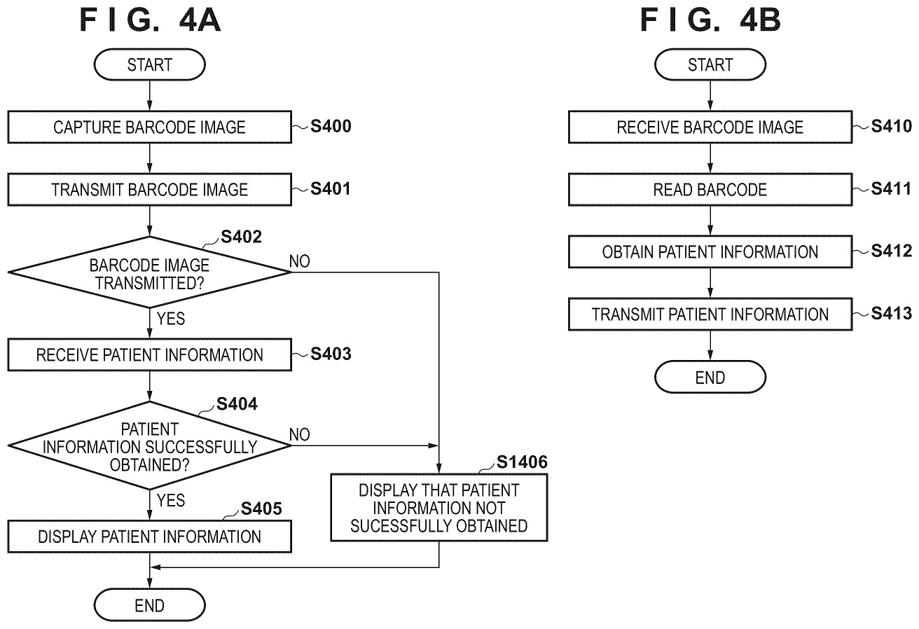

Next, processing in which the image capture apparatus 110 captures a barcode image and obtains patient information from the information management apparatus 120 will be described with reference to FIGS. 4A and 4B.

FIG. 4A indicates the processing of the image capture apparatus 110, and FIG. 4B indicates the processing of the information management apparatus 120.

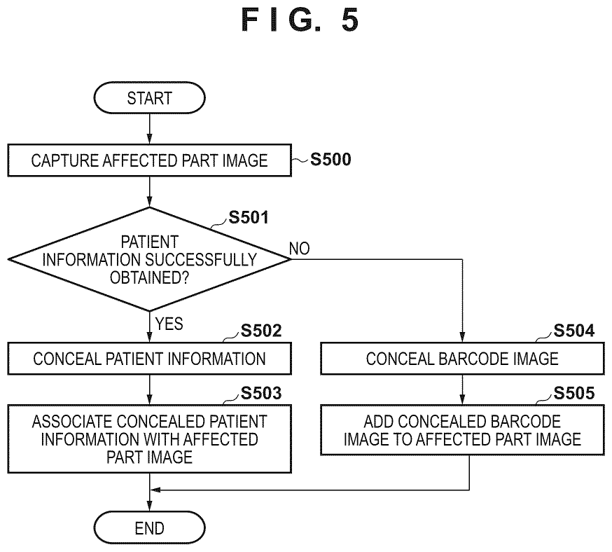

The processing of FIG. 4A is realized by the control unit 111 of the image capture apparatus 110 loading a program stored in the secondary storage unit 115 into the primary storage unit 114, executing the program, and controlling the respective components of the image capture apparatus 110. Further, the processing of FIG. 4B is realized by the control unit 121 of the information management apparatus 120 loading a program stored in the secondary storage unit 123 into the primary storage unit 122, executing the program, and controlling the respective components of the information management apparatus 120. It is similar for FIGS. 5, 12B, and 18, which will be described later.

In step S400, the control unit 111 of the image capture apparatus 110 captures a barcode image as in the screen 310 of FIG. 3 by controlling the optical unit 112 and the image capture unit 113 in response to an image capturing instruction from the user.

In step S401, the control unit 111 of the image capture apparatus 110 transmits the barcode image captured in step S400 to the information management apparatus 120.

In step S410, the control unit 121 of the information management apparatus 120 receives the barcode image transmitted from the image capture apparatus 110 in step S401.

In step S411, the control unit 121 of the information management apparatus 120 reads a patient ID from the barcode image received from the image capture apparatus 110 in step S410.

In step S412, the control unit 121 of the information management apparatus 120 searches through patient information stored in the secondary storage unit 123 and obtains patient information corresponding to the patient ID read from the barcode image. For example, since a patient ID read from the barcode 200 illustrated in FIG. 2A is “1234567”, the patient information 301 is obtained.

In step S413, the control unit 121 of the information management apparatus 120 transmits the patient information obtained in step S412 to the image capture apparatus 110.

In step S402, the control unit 111 of the image capture apparatus 110 determines whether the barcode image has been transmitted to the information management apparatus 120. When the control unit 111 of the image capture apparatus 110 determines that the barcode image has been transmitted to the information management apparatus 120, the processing proceeds to step S403. When the control unit 111 of the image capture apparatus 110 determines that the barcode image could not be transmitted to the information management apparatus 120, the processing proceeds to step S406. For example, when the communication environment is good, the barcode image can be transmitted to the information management apparatus 120, but when the communication environment is poor, transmission of the barcode image will fail.

In step S403, the control unit 111 of the image capture apparatus 110 receives the patient information from the information management apparatus 120.

In step S404, the control unit 111 of the image capture apparatus 110 determines whether the patient information has been successfully obtained and advances the processing to step S405 when it determines that the patient information has been successfully obtained and advances the processing to step S406 when it determines that the patient information has not been successfully obtained. For example, when the barcode image has not been captured properly, even when the barcode image is transmitted to the information management apparatus 120, the information management apparatus 120 will not be able to read the barcode image, and the patient information will not be successfully obtained.

In step S405, the control unit 111 of the image capture apparatus 110 displays the patient information received from the information management apparatus 120 on the display unit 117 as illustrated in the screen 320 of FIG. 3.

In this way, the image capture apparatus 110 can transmit the barcode image captured by the image capture apparatus 110 to the information management apparatus 120 and obtain the patient information corresponding to the barcode image from the information management apparatus 120.

In step S406, the control unit 111 of the image capture apparatus 110 notifies the user by displaying a message indicating that the barcode image could not be transmitted or that the patient information has not been successfully obtained on the display unit 117 as in the screen 340 of FIG. 3.

Next, processing in which the image capture apparatus 110 captures an affected part image after succeeding or failing to obtain patient information from the information management apparatus 120 will be described with reference to FIG. 5.

In step S500, the control unit 111 of the image capture apparatus 110 captures an affected part image as in the screen 330 or the screen 350 of FIG. 3 by controlling the optical unit 112 and the image capture unit 113 in response to an image capturing instruction from the user.

In step S501, the control unit 111 of the image capture apparatus 110 determines whether the patient information has been successfully obtained. The control unit 111 of the image capture apparatus 110 advances the processing to step S502 when it determines the patient information has been successfully obtained. The control unit 111 of the image capture apparatus 110 advances the processing to step S504 when it determines that the patient information has not been successfully obtained.

In step S502, the control unit 111 of the image capture apparatus 110 conceals the patient information obtained from the information management apparatus 120. Concealing may be, for example, encryption of information.

In step S503, the control unit 111 of the image capture apparatus 110 associates the patient information concealed in step S502 with the affected part image. The reason for concealing the patient information is, for example, to prevent the patient from being identified when the affected part image leaks to a third party.

In step S504, the control unit 111 of the image capture apparatus 110 conceals the barcode image captured in step S400 of FIG. 4A. The reason for concealing the barcode image is the same as the reason for concealing the patient information.

In step S505, the control unit 111 of the image capture apparatus 110 adds the barcode image concealed in step S504 to the affected part image.

In this way, the image capture apparatus 110 associates the affected part image and the patient information when the patient information has been successfully obtained and adds the barcode image to the affected part image when the patient information has not been successfully obtained.

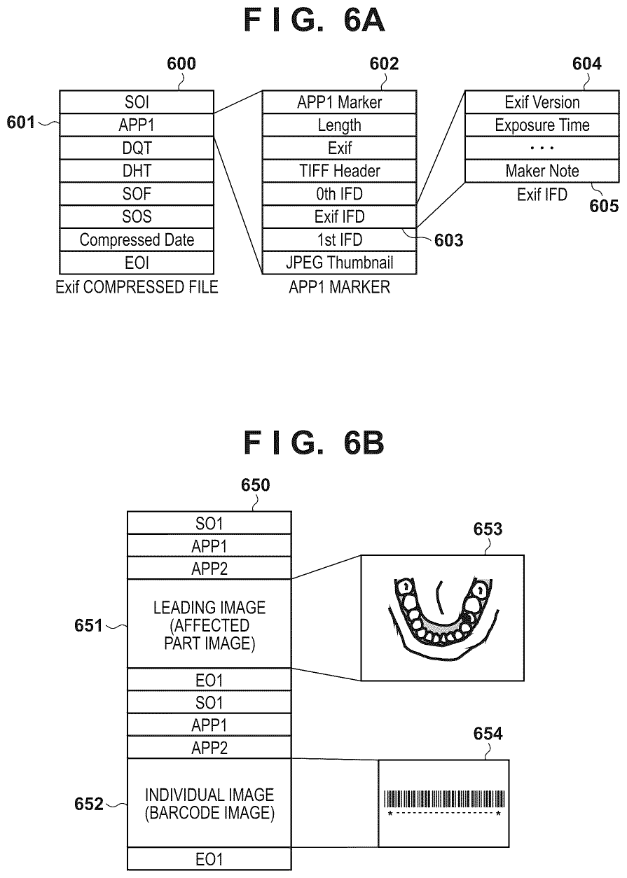

Next, a file format of an affected part image associated with concealed patient information will be described with reference to FIG. 6A. An image captured by the image capture apparatus 110 is a JPEG file in an Exif format. In the example of FIG. 6A, a structure of a file in the Exif format is illustrated. A file 600 in the Exif format includes a region 601 called APP1. The APP1 region 601 is a region for storing Exif attribute information. The APP1 region 601 has a data structure 602, and there is a region 603 called Exif IFD. The Exif IFD region 603 is a set of tags describing Exif-specific attribute information. The Exif IFD region 603 has a data structure 604, and there is a region 605 called Maker Note. The Maker Note region 605 stores items unique to the maker. The concealed patient information is stored in the Maker Note region 605 as it is information unique to the present embodiment.

Next, a file format of an affected part image to which concealed barcode image has been added will be described with reference to FIG. 6B. In the present embodiment, a barcode image is embedded in an affected part image in a multi-picture format. In the multi-picture format, a plurality of pieces of image data can be recorded as one file. FIG. 6B illustrates a structure 650 of a file in the multi-picture format. In the multi-picture format, the structure is such that SOI to EOI are repeated for the number of image files to be recorded. In the example of FIG. 6B, an affected part image 653 is stored in a leading image region 651, and a barcode image 654 is stored in an individual image region 652. By storing the barcode image in the affected part image as described above, even when patient information has not been successfully obtained, information (patient ID) necessary for obtaining patient information can be obtained from the affected part image.

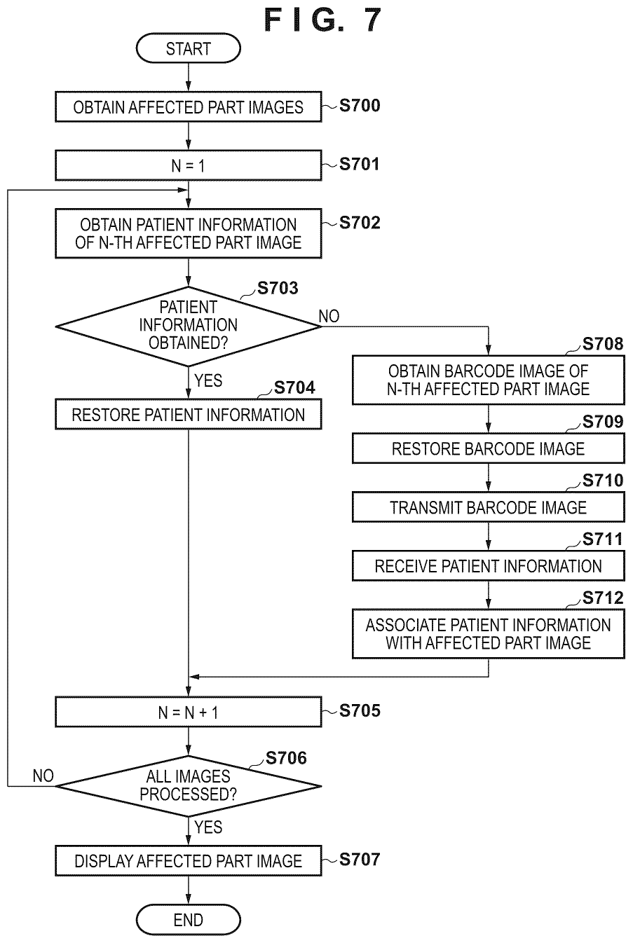

Next, processing in which the image processing apparatus 100 receives an affected part image from the image capture apparatus 110 and associates patient information received from the information management apparatus 120 and the affected part image will be described with reference to FIG. 7.

In the present embodiment, the image processing apparatus 100 and the image capture apparatus 110 are connected by a wire, such as a USB cable. Further, the image processing apparatus 100 and the information management apparatus 120 are connected by a wire via a network in a medical facility. It is similar for second and third embodiments to be described later.

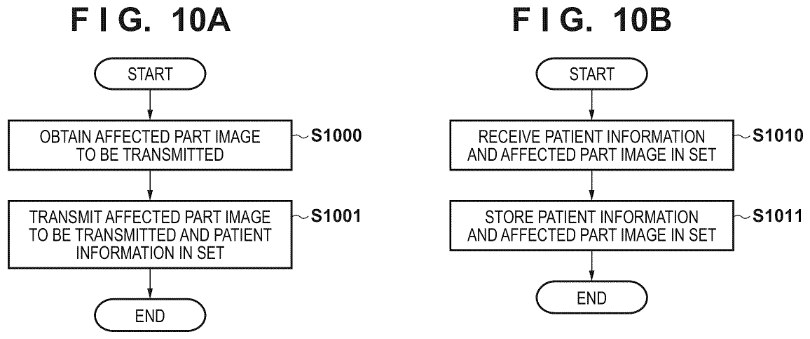

The processing of FIG. 7 is realized by the control unit 101 of the image processing apparatus 100 loading a program stored in the secondary storage unit 105 into the primary storage unit 104, executing the program, and controlling the respective components of the image processing apparatus 100. It is similar for FIGS. 10A, 12A, 13, and 17, which will be described later.

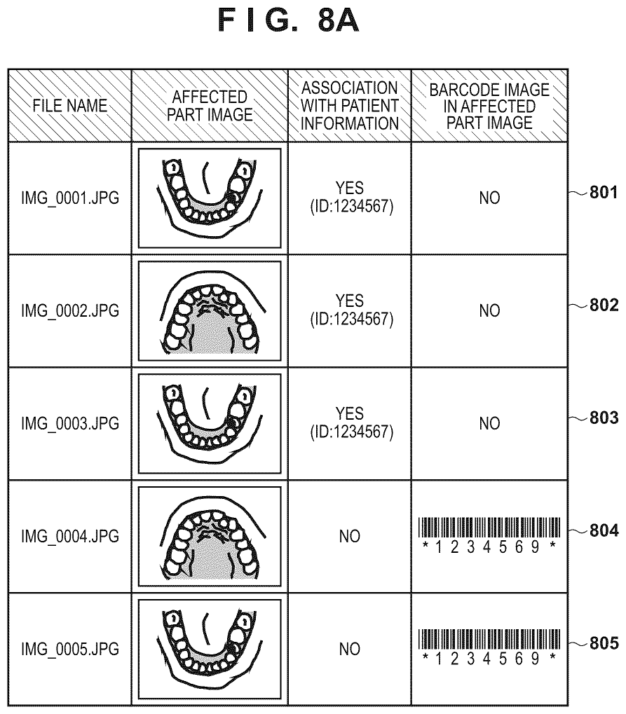

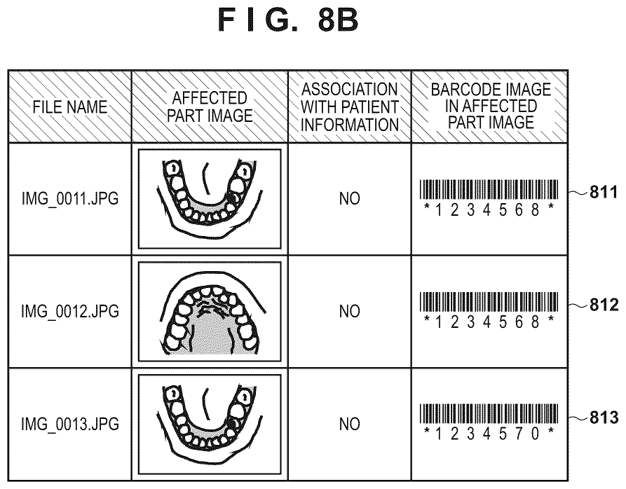

In step S700, the control unit 101 obtains affected part images from the image capture apparatus 110. Five affected part images 801 to 805 illustrated in FIG. 8A are stored in the image capture apparatus 110 and transmitted to the image processing apparatus 100. Here, steps S702 to S706 and steps S708 to S711 of FIG. 7 are processes repeated for the number of affected part images received from the image capture apparatus 110.

In step S701, the control unit 101 sets the value of N to 1. The value of N is a value representing the index of an image. That is, when N is 1, a first image is processed.

In step S702, the control unit 101 obtains patient information of a first affected part image. In the example of FIG. 8A, a first image 801 is associated with patient information, and a patient ID is “1234567”.

In step S703, the control unit 101 determines whether patient information has been obtained, and when patient information has been obtained as for the affected part image 801 of FIG. 8A, the processing proceeds to step S704. When patient information is not associated as in an affected part image 804 of FIG. 8A, the control unit 101 advances the processing to step S708.

In step S704, the control unit 101 restores the concealed patient information. Restoration is, for example, decryption of encrypted information.

In step S705, the control unit 101 increments the value of N.

In step S706, the control unit 101 determines whether all of the affected part images received in step S700 have been processed. The control unit 101 advances the processing to step S707 when it determines that all of the affected part images received in step S700 have been processed. The control unit 101 returns the processing to step S702 when it determines that all of the affected part images received in step S700 have not been processed. As illustrated in FIG. 8A, five affected part images 801 to 805 are stored in the image capture apparatus 110, and so, the processing returns to step S702 at this time, but the affected part images 801 to 803 are associated with patient information, and so, the processing from steps S702 to S705 is repeated. Then, when processing the fourth image 804, since the image 804 is not associated with patient information and a barcode image has been added instead, the processing of step S708 is executed.

In step S708, the control unit 101 obtains a barcode image added to the affected part image.

In step S709, the control unit 101 restores the concealed barcode image obtained in step S708.

In step S710, the control unit 101 transmits the barcode image restored in step S709 to the information management apparatus 120. The method of transmitting a barcode image from the image capture apparatus 110 to the information management apparatus 120 is wireless communication, and so, the image may not be successfully transmitted when the communication environment is poor, but the image processing apparatus 100 and the information management apparatus 120 are connected by a wire, and so, the possibility that transmission of the image will fail is reduced. The processing for receiving a barcode image and searching for patient information in the information management apparatus 120 is similar to the processing of FIG. 4B.

In step S711, the control unit 101 receives patient information from the information management apparatus 120.

In this way, it is possible to obtain patient information using a barcode image added to an affected part image even when the affected part image is not associated with patient information.

In step S712, the control unit 101 associates the patient information received from the information management apparatus 120 with the affected part image. The processing for associating patient information with an affected part image need not only be configured such that the patient information is associated with the affected part image as in the image capture apparatus 110 but may also be configured such that the affected part image and the patient information can be transmitted in a set. That is, the affected part image and the patient information need only be associated with each other in the image processing apparatus 100. The control unit 101 advances the processing to step S707 when all of the images of the image capture apparatus 110 have been processed.

In step S707, the control unit 101 displays all of the affected part images associated with patient information on the display unit 102. In the processing of FIG. 7, the image processing apparatus 100 restores patient information or a barcode image, but a concealed patient image or a concealed barcode image may be transmitted to the information management apparatus 120 and restored in the information management apparatus 120.

Next, an application screen in which affected part images are displayed in a list in step S707 of FIG. 7 will be described with reference to FIG. 9A.

The image management application groups and displays images having the same patient information as indicated in a screen 900. In the example of FIG. 9A, affected part images associated with patient information 903 are grouped into a group 901, and affected part images associated with patient information 904 are grouped into a group 902. A button 905 is a button with which patient information can be changed and is operated at the time of changing the association between an affected part image and patient information, such as when an affected part image and patient information are incorrectly associated with each other.

Further, an affected part image to be transmitted to the information management apparatus 120 can be selected with a check box 906. The check boxes are all checked by default, and when there is an affected part image that does not need to be transmitted to the information management apparatus 120, that affected part image can be excluded from being a target of transmission to the information management apparatus 120 by unchecking the check box of that affected part image. A transmission button 907 is a button for executing processing for transmitting an affected part image to the information management apparatus 120, and the user can transmit an affected part image whose check box is checked to the information management apparatus 120 by operating the image transmission button 907.

Next, processing in which the image processing apparatus 100 transmits an affected part image and patient information to the information management apparatus 120 will be described with reference to FIGS. 10A and 10B.

FIG. 10A indicates the processing of the image processing apparatus 100, and FIG. 10B indicates the processing of the information management apparatus 120.

In step S1000, the control unit 101 of the image processing apparatus 100 obtains a transmission target affected part image to be uploaded to the information management apparatus 120. The transmission target affected part image is an affected part image for which the check box 906 has been checked in the application screen 900 of FIG. 9A.

In step S1001, the control unit 101 of the image processing apparatus 100 transmits the affected part image to be transmitted and patient information associated with that affected part image in a set to the information management apparatus 120.

In step S1010, the control unit 121 of the information management apparatus 120 receives the affected part image and the patient information transmitted from the image processing apparatus 100, in a set.

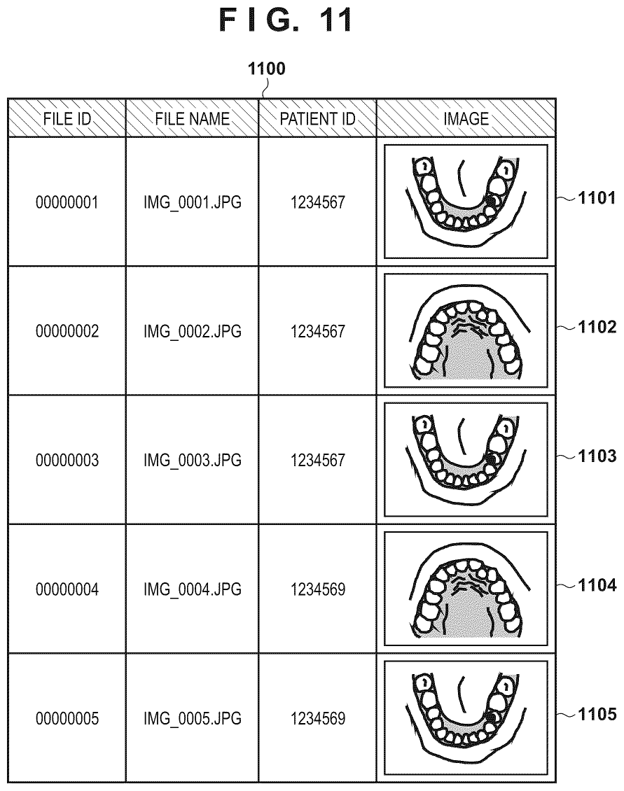

In step S1011, the control unit 121 of the information management apparatus 120 stores the affected part image and the patient information received from the image processing apparatus 100, in a set. FIG. 11 illustrates affected part images and patient information stored in sets in the information management apparatus 120. When the information management apparatus 120 stores affected part images, the images are stored in association with patient information. In the example of FIG. 11, the patient IDs of affected part images 1101 to 1103 are “1234567”. Further, the patient IDs of affected part images 1104 and 1105 are “1234569”. As described above, by storing patient information and an affected part image in association with each other, it is possible to search only for affected part image whose patient ID is “1234567”, for example. In the example of FIG. 11, a file ID, a file name, and a patient ID are associated with an affected part image, but information such as an image capturing date and time and a date and time of transmission to the information management apparatus 120 may be associated.

Next, an application screen in which affected part images are displayed in a list in step S707 of FIG. 7 will be described with reference to FIG. 9B.

When the image capture apparatus 110 has obtained patient information from the information management apparatus 120, the image capture apparatus 110 associates an affected part image with the patient information. When the image capture apparatus 110 cannot obtain patient information, the image processing apparatus 100 obtains patient information from the information management apparatus 120 and associates an affected part image with the patient information.

FIG. 9B illustrates a screen in which an icon indicating in which of the image capture apparatus 110 and the image processing apparatus 100 the association has been performed is displayed, in contrast to the screen 900 of FIG. 9A. In the example of FIG. 9B, icons 913 of the image capture apparatus indicating that association has been performed in the image capture apparatus 110 for affected part images grouped into a group 911 are displayed. Further, icons 914 of the image processing apparatus 100 indicating that association has been performed in the image processing apparatus 100 for affected part images grouped into a group 912 are displayed.

Next, a case where the information management apparatus 120 has failed to read a barcode image and the image capture apparatus 110 cannot obtain patient information from the information management apparatus 120 will be described.

For example, when a barcode image could not be properly captured by the image capture apparatus 110, the information management apparatus 120 will fail to read the barcode image, and the image capture apparatus 110 cannot obtain patient information from the information management apparatus 120. FIG. 8B illustrates affected part images and barcode images stored in the image capture apparatus 110 when the information management apparatus 120 has failed to read the barcode images and cannot obtain patient information from the information management apparatus 120 and the image capture apparatus 110 continues capturing the affected part images. In the example of FIG. 8B, three affected part images 811 to 813 are stored in the image capture apparatus 110, and all of the affected part images are not associated with patient information. Further, although barcode images corresponding to the affected part images 811 and 812 are properly captured, a barcode image corresponding to the image 813 is slightly blurred and is not properly captured. The information management apparatus 120 will fail to read such a barcode image that has not been properly captured, and patient information cannot be obtained. FIG. 9C illustrates an application screen for when the information management apparatus 120 has failed to read a barcode image and the image capture apparatus 110 cannot obtain patient information from the information management apparatus 120 will be described. In the example of FIG. 9C, patient information 925 has been obtained from the information management apparatus 120 for two affected part images grouped into a group 921, but patient information has not been obtained for an affected part image grouped into a group 922. In such a case, by the user directly inputting a patient ID and the inputted patient ID being transmitted to the information management apparatus 120, the information management apparatus 120 can search for patient information. For example, when the user enters a patient ID in a text field 926 of a screen 920 of FIG. 9C and operates an “OK” button 927, the patient ID is transmitted to the information management apparatus 120, and the information management apparatus 120 searches for patient information. Further, a barcode image 924 is displayed adjacent to an affected part image 923 such that the user can directly enter a patient ID. When a barcode is only slightly blurred, the user can visually confirm and enter a patient ID from the barcode image.

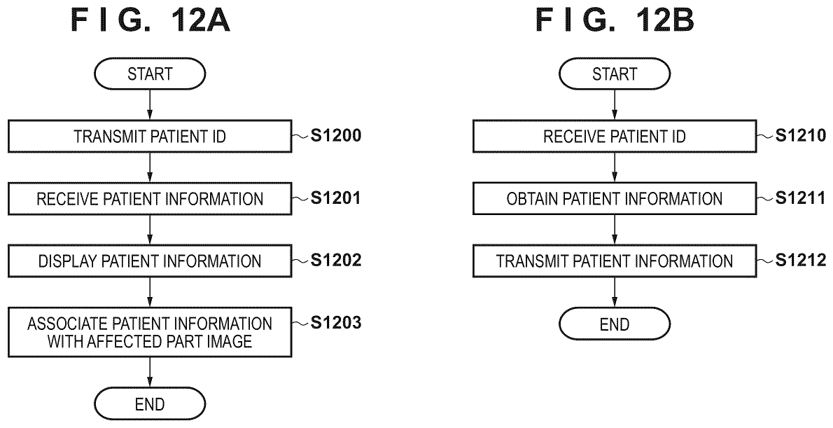

FIGS. 12A and 12B are flowcharts illustrating processing in which the image processing apparatus 100 transmits a patient ID to the information management apparatus 120 and the information management apparatus 120 searches for patient information.

FIG. 12A indicates the processing of the image processing apparatus 100, and FIG. 12B indicates the processing of the information management apparatus 120.

In step S1200, the control unit 101 of the image processing apparatus 100 transmits a patient ID inputted to the application screen 920 of FIG. 9C to the information management apparatus 120.

In step S1210, the control unit 121 of the information management apparatus 120 receives the patient ID from the image processing apparatus 100.

In step S1211, the control unit 121 of the information management apparatus 120 searches through patient information stored in the information management apparatus 120 and obtains patient information corresponding to the patient ID received from the image processing apparatus 100 in step S1210.

In step S1212, the control unit 121 of the information management apparatus 120 transmits the patient information obtained in step S1211 to the image processing apparatus 100.

In step S1201, the control unit 101 of the image processing apparatus 100 receives the patient information from the information management apparatus 120.

In step S1202, the control unit 101 of the image processing apparatus 100 displays the patient information received from the information management apparatus 120 in step S1201 on an application screen. FIG. 9D illustrates the application screen displayed in step S1202. In the example of FIG. 9D, patient information 931 obtained from the information management apparatus 120 by inputting a patient ID is displayed on an application screen 930, in contrast to the example of FIG. 9C.

In step S1203, the control unit 101 of the image processing apparatus 100 associates the patient information received from the information management apparatus 120 in step S1201 with the affected part image.

In this way, even when the information management apparatus 120 fails to read a barcode image, it is possible to obtain patient information from the information management apparatus 120 by inputting a patient ID and associating the patient information with an affected part image.

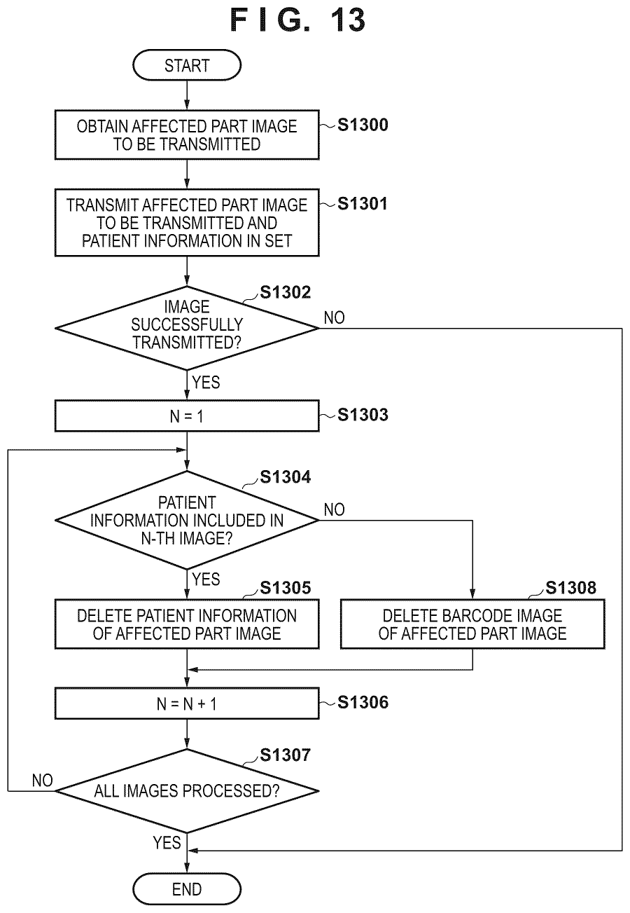

Next, processing in which the image processing apparatus 100 deletes patient information associated with an affected part image or patient information added to an affected part image after transmitting the affected part image and the patient information to the information management apparatus 120 will be described with reference to FIG. 13.

The processing of steps S1300 and S1301 of FIG. 13 is the same processing as that of steps S1000 and S1001 of FIG. 10A.

In step S1302, the control unit 101 determines whether an affected part image and patient information have been successfully transmitted and advances the processing to step S1303 when it determines that they have been successfully transmitted and ends the processing when it determines that they have not been successfully transmitted.

In step S1303, the control unit 101 sets the value of N to 1.

In step S1304, the control unit 101 determines whether patient information is included in the affected part image and advances the processing to step S1305 when it determines that patient information is included and advances the processing to step S1308 when it determines that patient information is not included.

In step S1305, the control unit 101 deletes the patient information associated with the affected part image.

In step S1306, the control unit 101 increments the value of N.

In step S1307, the control unit 101 determines whether all of the affected part images received from the image capture apparatus 110 have been processed and ends the processing when it determines that all of the affected part images have been processed and returns the processing to step S1304 when it determines that all the affected part images have not been processed. In step S1304, when the patient information is not included in the affected part image, it means that a barcode image is included instead.

In step S1308, the control unit 101 deletes the barcode image added to the affected part image.

In this way, it is possible for the image processing apparatus 100 to delete patient information associated with an affected part image or patient information added to an affected part image after transmitting the affected part image and the patient information to the information management apparatus 120.

When deleting the barcode image added to the affected part image in step S1308, the barcode image may be replaced with a reduced image (thumbnail) of the affected part image.

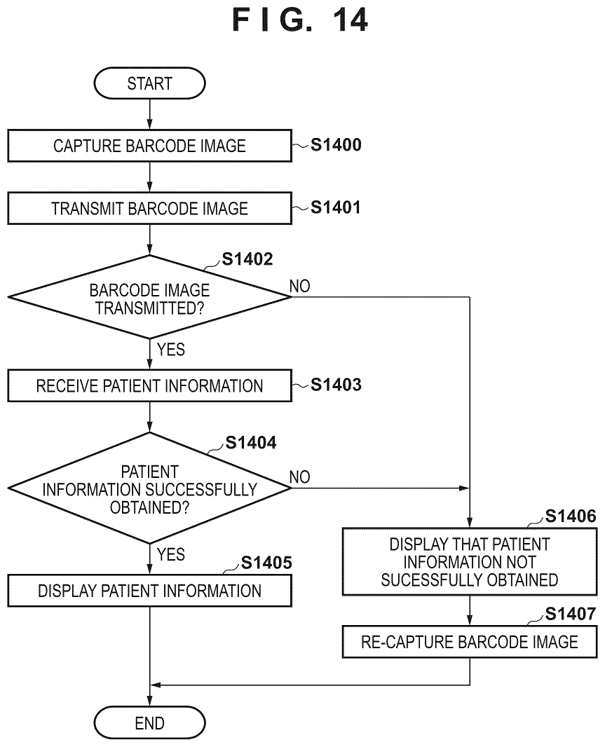

Next, processing in which the image capture apparatus 110 re-captures a barcode image when it has failed to obtain patient information from the information management apparatus 120 will be described with reference to FIG. 14.

The processing of steps S1400 to S1406 of FIG. 14 is the same processing as that of steps S400 to S406 of FIG. 4A.

When the control unit 111 fails to obtain patient information from the information management apparatus 120 in step S1404, the control unit 111 advances the processing to step S1406 and prompts the user to re-capture a barcode image. In the present embodiment, when the image capture apparatus 110 cannot obtain patient information from the information management apparatus 120, the image processing apparatus 100 obtains patient information from the information management apparatus 120 using a barcode image obtained from the image capture apparatus 110; a plurality of barcode images are captured in case the image processing apparatus 100 fails to read a barcode image. The plurality of barcode images are embedded in an affected part image in a multi-picture format.

### Second Embodiment

In a second embodiment, processing in which the image capture apparatus 110 stores a barcode image and an affected part image in a distinguishable manner as separate files without adding the barcode image to the affected part image when it fails to obtain the patient information from the information management apparatus 120 will be described.

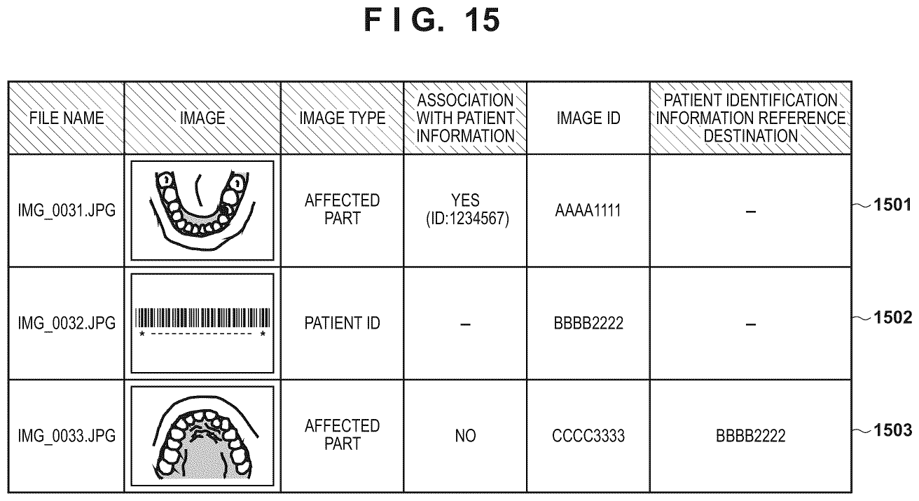

FIG. 15 illustrates affected part images stored in the image capture apparatus 110 according to the second embodiment. In the example of FIG. 15, three affected part images 1501 to 1503 are stored. One difference from the first embodiment is that “image type”, “image ID”, and “patient identification information reference destination” are added to attribute information of the affected part images 1501 to 1503. The “image type” is information for distinguishing whether the image is an affected part image or a barcode image. The “image ID” is information that uniquely identifies the image. The “patient identification information reference destination” is information stored only for an affected part image not associated with patient information and is information indicating the image ID of a barcode image. In the example of FIG. 15, the affected part image 1501 is associated with patient information, but the affected part image 1503 is not associated with patient information. Regarding such an affected part image not associated with patient information, a corresponding barcode image is stored as a separate file. The patient identification information reference destination for the affected part image 1503 is “BBBB2222”, and so, the image 1502 is a corresponding barcode image.

FIG. 16 illustrates an application screen in which affected part images received from the image capture apparatus 110 storing the affected part images illustrated in FIG. 15 are displayed on the image processing apparatus 100. In an application screen 1600, two affected part images are displayed in groups 1601 and 1602, and patient information is displayed for each affected part image.

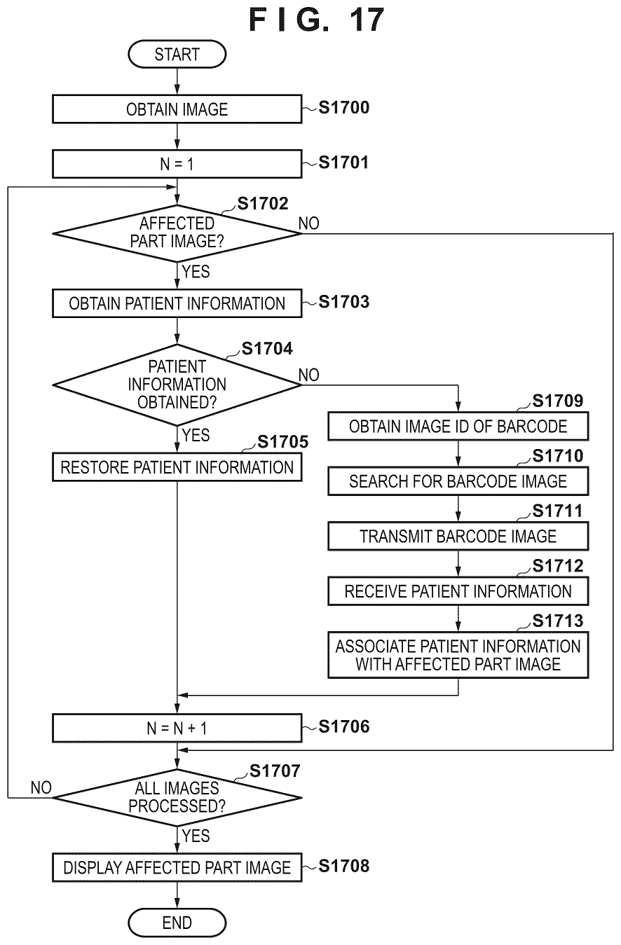

Next, processing in which the image processing apparatus 100 receives an affected part image from the image capture apparatus 110 and associates patient information received from the information management apparatus 120 and the affected part image will be described with reference to FIG. 17.

The processing of steps S1700 and S1701 of FIG. 17 is the same processing as that of steps S700 and S701 of FIG. 7.

In step S1702, the control unit 101 determines whether an image to be processed is an affected part image and advances the processing to step S1703 when it determines that the image is an affected part image and advances the processing to step S1707 when it determines that the image is not an affected part image.

The processing of steps S1703 to S1707 is the same processing as that of steps S702 to S706 of FIG. 7.

In step S1709, the control unit 101 obtains the image ID of a barcode image corresponding to the affected part image. In a case of the affected part image 1503 of FIG. 15, “BBBB2222” is obtained.

In step S1710, the control unit 101 searches for a barcode image corresponding to the image ID obtained in step S1709. In the example of FIG. 15, the image 1502 is obtained.

In this way, in the second embodiment, it is possible to obtain patient information using a barcode image stored as a file separate from an affected part image.

The processing of steps S1711 to S1713 of FIG. 17 is the same processing as that of steps S710 to S712 of FIG. 7.

In step S1707, when all of the images have been processed, the processing proceeds to step S1708, and the control unit 101 displays all of the affected part images associated with patient information in a list, as illustrated in FIG. 16.

When patient information has been successfully obtained from a barcode image in step S1712, the barcode image transmitted to the information management apparatus 120 may be deleted.

### Third Embodiment

In a third embodiment, the processing of the image capture apparatus 110 is simplified by always adding a barcode image to an affected part image regardless of whether the image capture apparatus 110 has succeeded or failed to obtain patient information from the information management apparatus 120.

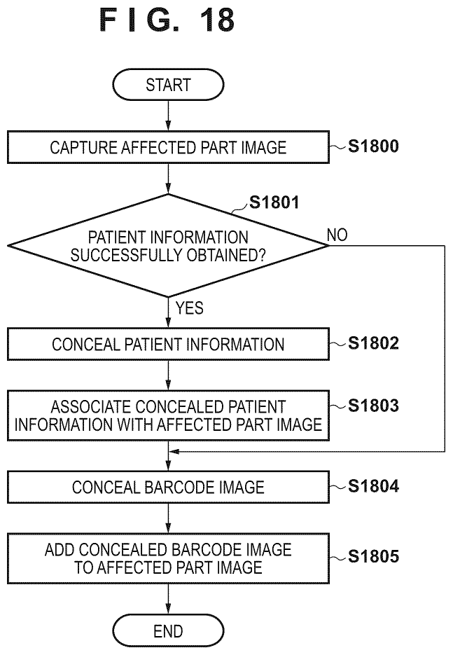

FIG. 18 illustrates processing in which the image capture apparatus 110 captures an affected part image and adds a barcode image to the captured affected part image.

The processing of steps S1800 to S1803 is the same processing as that of steps S500 to S503 of FIG. 5.

In the first embodiment, a barcode image is added to an affected part image only when patient information has not been successfully obtained, but in the third embodiment, a barcode image is always added to an affected part image regardless of whether patient information has been successfully obtained. Therefore, even when patient information has been successfully obtained in step S1801, a barcode image is added to the affected part image in steps S1804 and S1805 after associating the patient information with the affected part image in steps S1802 and S1803.

The processing of steps S1804 and S1805 is the same processing as that of steps S504 and S505 of FIG. 5.

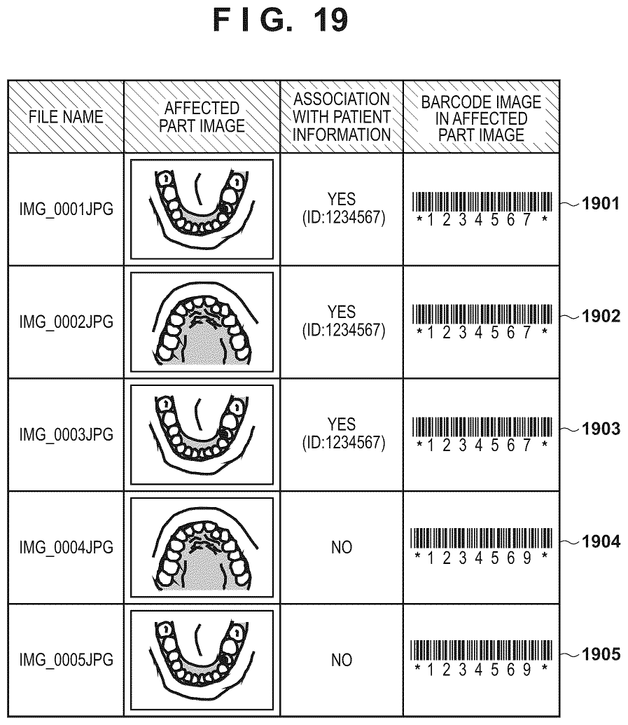

FIG. 19 illustrates a state in which a barcode image has been added to all of the affected part images. In the example of FIG. 19, a barcode image has been added to all of five images 1901 to 1905, and three images 1901 to 1903 are associated with patient information, and two images 1904 and 1905 are not associated with patient information.

As described above, by adding a barcode image to all of the affected part images, it is possible to simplify the processing of the image capture apparatus 110 and confirm whether patient information is incorrect by displaying the barcode images added to the affected part images. In this case, by providing a button for displaying a barcode image on an application screen displaying affected part images in a list, the user can confirm a barcode image corresponding to an affected part image at any time.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2023-093305, filed Jun. 6, 2023 which is hereby incorporated by reference herein in its entirety.

What is claimed is:

1. An image processing apparatus comprising:
an obtaining unit that obtains, from a first external apparatus, a first image in which a predetermined part of a patient has been captured; and
a control unit that:
in a case where information of the patient cannot be obtained from the first image, transmits, to a second external apparatus, a second image for identifying a patient, the second image having been added to the first image, and
obtains, from the second external apparatus, information of the patient corresponding to the second image and associate the information with the first image.

2. The apparatus according to claim 1, wherein
the information of the patient associated with the first image or the second image added to the first image is concealed, and
the control unit restores the concealed information of the patient or the concealed second image.

3. The apparatus according to claim 1, further comprising:
a display unit that displays the first image obtained from the first external apparatus,
wherein in a case where the first image obtained from the first external apparatus is displayed, the control unit displays the information of the patient associated with the first image, or displays the second image added to the first image and performs display so as to allow input of information for identifying the patient.

4. The apparatus according to claim 3, wherein
the display unit displays that the first image has been associated with the information of the patient in the first external apparatus or the first image has been associated with the information of the patient in the image processing apparatus.

5. The apparatus according to claim 1, wherein
the control unit displays the first image to be transmitted to the second external apparatus so as to be selectable.

6. The apparatus according to claim 3, wherein
the control unit displays the information of the patient and the first image or the second image, which has been grouped for each piece of information of the patient.

7. The apparatus according to claim 3, wherein
the control unit displays the information of the patient so as to be changeable.

8. The apparatus according to claim 7, wherein
the control unit:
transmits identification information for identifying the patient to the second external apparatus, and
associates the information of the patient received from the second external apparatus with the first image.

9. The apparatus according to claim 1, wherein
the control unit deletes the information of the patient associated with the first image or the second image added to the first image after transmitting the first image and the information of the patient associated with the first image to the second external apparatus.

10. The apparatus according to claim 1, wherein
information of a destination for referencing the second image is added to the first image obtained from the first external apparatus.

11. The apparatus according to claim 1, wherein
the first image is an image in which an affected part of the patient has been captured, and
the second image is an image in which a barcode to which identification information for identifying the patient has been added has been captured.

12. The apparatus according to claim 11, wherein
the second external apparatus:
stores the information of the patient including the identification information, and
reads the identification information from the second image received from the image processing apparatus and transmits the information of the patient corresponding to the identification information to the image processing apparatus.

13. The apparatus according to claim 1, wherein
the second external apparatus:
receives the first image and the information of the patient associated with the first image from the image processing apparatus, and
stores the first image and the information of the patient received from the image processing apparatus.

14. An image capture apparatus comprising:
an image capture unit that captures a first image indicating a predetermined part of a patient and a second image for identifying a patient;
a communication unit that transmits the second image to an external apparatus; and
a control unit that:
in a case where information of the patient corresponding to the second image has been obtained from the external apparatus, associates the information of the patient with the first image, and
in a case where the information of the patient corresponding to the second image cannot be obtained from the external apparatus, adds the second image to the first image.

15. The apparatus according to claim 14, wherein
the control unit:
in a case where the information of the patient has been obtained from the external apparatus, displays the information of the patient and inquires a user whether to capture the first image, and
in a case where the information of the patient cannot be obtained from the external apparatus, displays that the information of the patient cannot be obtained and inquires the user whether to capture the first image.

16. The apparatus according to claim 14, wherein
the control unit:
in a case where the information of the patient has been obtained from the external apparatus, conceals the information of the patient and associates the information of the patient with the first image, and
in a case where the information of the patient cannot be obtained from the external apparatus, conceals the second image and adds the second image to the first image.

17. The apparatus according to claim 14, wherein
in a case where the information of the patient cannot be obtained from the external apparatus, the control unit inquires a user to re-capture the second image.

18. The apparatus according to claim 14, wherein
the control unit stores the second image and the first image as separate files, and
the control unit adds information of a destination for referencing the second image to the first image not associated with the information of the patient.

19. The apparatus according to claim 14, wherein
even in a case where the information of the patient has been obtained from the external apparatus, the control unit adds the second image to the first image.

20. The apparatus according to claim 14, wherein
the first image is an image in which an affected part of the patient has been captured, and
the second image is an image in which a barcode to which identification information of the patient has been added has been captured.

21. The apparatus according to claim 20, wherein
the external apparatus:
stores the information of the patient including the identification information of the patient, and
reads the identification information of the patient from the second image received from the image capture apparatus and transmits the information of the patient corresponding to the identification information of the patient to the image capture apparatus.

22. A system including:
an image capture apparatus that captures a first image indicating a predetermined part of a patient and a second image for identifying a patient;
an image processing apparatus that obtains the first image to which the second image has been added from the image capture apparatus; and
an information management apparatus that reads identification information of the patient from the second image received from the image capture apparatus or the image processing apparatus and transmits information of the patient corresponding to the identification information to the image capture apparatus or the image processing apparatus.

23. A method of controlling an image processing apparatus, the method comprising:
obtaining, from a first external apparatus, a first image in which a predetermined part of a patient has been captured; and
in a case where information of the patient cannot be obtained from the first image, transmitting, to a second external apparatus, a second image for identifying a patient, the second image having been added to the first image, and
obtaining, from the second external apparatus, information of the patient corresponding to the second image and associating the information with the first image.

24. A method of controlling an image capture apparatus, the method comprising:
capturing a second image for identifying a patient;
transmitting the second image to an external apparatus; and
in a case where information of the patient corresponding to the second image has been obtained from the external apparatus, associating the information of the patient with a first image in which the patient has been captured, and
in a case where the information of the patient corresponding to the second image cannot be obtained from the external apparatus, adding the second image to the first image.

25. A non-transitory computer-readable storage medium storing a program for causing a processor to function as an image processing apparatus comprising:
an obtaining unit that obtains, from a first external apparatus, a first image in which a predetermined part of a patient has been captured; and
a control unit that:
in a case where information of the patient cannot be obtained from the first image, transmits, to a second external apparatus, a second image for identifying a patient, the second image having been added to the first image, and
obtains, from the second external apparatus, information of the patient corresponding to the second image and associate the information with the first image.

26. A non-transitory computer-readable storage medium storing a program for causing a processor to function as an image capture apparatus comprising:
an image capture unit that captures a first image indicating a predetermined part of a patient and a second image for identifying a patient;
a communication unit that transmits the second image to an external apparatus; and
a control unit that:
in a case where information of the patient corresponding to the second image has been obtained from the external apparatus, associates the information of the patient with the first image, and
in a case where the information of the patient corresponding to the second image cannot be obtained from the external apparatus, adds the second image to the first image.
